(19) United States
(12) Patent Application Publication
(10) Pub. No.: US 2025/0228615 A1
(43) Pub. Date: Jul. 17, 2025

(54) SYSTEMS AND METHODS FOR TRANSCATHETER SURGERY

(71) Applicants: Children's Medical Center Corporation, Boston, MA (US); The General Hospital Corporation, Boston, MA (US)

(72) Inventors: Pierre E. Dupont, Boston, MA (US); Sammy Elmariah, Corte Madera, CA (US)

(73) Assignees: Children's Medical Center Corporation, Boston, MA (US); The General Hospital Corporation, Boston, MA (US)

(21) Appl. No.: 18/703,198

(22) PCT Filed: Oct. 19, 2022
(86) PCT No.: PCT/US2022/078352

§ 371 Date: Apr. 19, 2024

Related U.S. Application Data
(60) Provisional application No. 63/257,451, filed on Oct. 19, 2021.

(51) Int. Cl. A61B 18/24; A61B 18/00; A61B 18/14; A61B 18/20; A61B 18/22

(52) U.S. Cl. CPC A61B 18/24; A61B 18/1492; A61B 2018/0022; A61B 2018/00369; A61B 2018/00601; A61B 2018/1467; A61B 2018/202; A61B 2018/2211; A61B 2018/2222; A61B 2018/2253

(57) ABSTRACT

A system for lacerating a tissue includes a catheter including one or more lacerators, where the one or more lacerators are configured to lacerate the tissue at a first exposure window and a second exposure window. The system also includes one or more aligners, where the one or more aligners are deployable and configured to, when deployed, promote contact between the one or more lacerators and the tissue at the first exposure window and/or the second exposure window.

### CROSS-REFERENCE TO RELATED APPLICATIONS

This application claims the benefit under 35 U.S.C. § 119(e) of U.S. Provisional Patent Application No. 63/257,451 filed Oct. 19, 2021, the contents of which application are incorporated herein by reference in their entirety.

### BACKGROUND OF TECHNOLOGY

Transcatheter heart valve repair and replacement have revolutionized care of the heart failure patient who is too sick to undergo surgery. With an increased experience base, the efficacy and safety of these treatments is enabling them to be extended to younger and healthier patients.

This younger patient population presents new challenges, however, since they can outlive the implanted devices they receive. While surgical re-operations facilitate removal of previously implanted devices so that they do not interfere with new repairs and devices, this is not straightforward for transcatheter procedures. And due to device endothelialization, which is often desirable to minimize long-term inflammatory response, it is difficult to design devices to be easily removable after chronic implantation. Consequently, new techniques and tools are needed to perform surgery so that native tissue as well as previously implanted devices can be reliably modified or removed to make way for a new repair.

A clinically important class of such problems requires the cutting of valve leaflets. For example, in transcatheter aortic valve replacement (TAVR), the new valve may cause the existing native or bioprosthetic leaflets to occlude the coronary arteries. To prevent occlusion, the existing leaflets must be sliced down the middle or removed. As a second example, in transcatheter mitral valve replacement, to ensure that the left ventricular outflow tract remains clear, it is sometimes necessary to remove part of native anterior leaflet. Or if the valve had previously been repaired, the anterior leaflet must be cut such that the clips remain attached to the posterior leaflet and are pressed against the left ventricular free wall by the new valve.

A substantial challenge to cutting tissue inside the heart is that the tissue includes calcified deposits. Such calcifications of leaflet and annulus tissue are often the cause of the valve stenosis that needs to be repaired. Several groups have proposed catheter-delivered shockwaves to break up the calcium deposits in valve leaflets. Similarly, calcified lesions in and around the coronary arteries lead to stenosis as well as in-stent restenosis. Some techniques use a fluid-filled balloon with shockwave generator to break up calcium in tissue surrounding vessel to enable balloon dilation. Excimer lasers are used to open occluded vessels in moderately calcified lesions.

### SUMMARY

The present disclosure relates to a system for lacerating a tissue, including: a catheter including one or more lacerators, wherein the one or more lacerators are configured to lacerate the tissue at a first exposure window and a second exposure window; and one or more aligners, wherein the one or more aligners are deployable and configured to, when deployed, promote contact between the one or more lacerators and the tissue at the first exposure window and/or the second exposure window.

In some embodiments, the present disclosure relates to a system, wherein: the first exposure window is arranged in the catheter to enable a lacerator of the one or more lacerators to pierce the tissue to create an opening in the tissue; and a first aligner of the one or more aligners is arranged to, when deployed, promote contact between the tissue and the second exposure window when at least a part of the catheter is disposed in the opening of the tissue. In some embodiments, the present disclosure relates to a system, wherein: a second aligner of the one or more aligners is arranged to, when deployed, promote positioning of the catheter with respect to the tissue for piercing of the tissue by the lacerator. In some embodiments, the present disclosure relates to a system, wherein the second aligner is positioned on an opposite side of the tissue from the first aligner, when the first aligner is deployed. In some embodiments, the present disclosure relates to a system, wherein the first exposure window and the second exposure window are contiguous. In some embodiments, the present disclosure relates to a system, further including: an energy source couplable to at least one of the one or more lacerators and configured to be activated to transmit energy to the tissue, the transmitted energy passing towards the tissue through the at least one of the one or more lacerators at the first exposure window and/or the second exposure window. In some embodiments, the present disclosure relates to a system, wherein: the at least one of the one or more lacerators includes an optical fiber; and the energy source is a laser source configured to transmit laser energy to the tissue, the transmitted laser energy passing through the optical fiber towards the tissue at the first exposure window and/or the second exposure window to lacerate the tissue. In some embodiments, the present disclosure relates to a system, wherein: the at least one of the one or more lacerators is one or more electrodes; and the energy source is an electrosurgical energy source configured to transmit electrosurgical energy to the tissue, the transmitted electrosurgical energy passing from or between the one or more electrodes through the tissue at the first exposure window and/or the second exposure window to lacerate the tissue. In some embodiments, the present disclosure relates to a system, wherein: the catheter includes at least one of the one or more aligners; the first exposure window is arranged in the catheter distal to the second exposure window; and the second exposure window is arranged in the catheter distal to the at least one of the one or more aligners. In some embodiments, the present disclosure relates to a system, wherein: the catheter includes at least one of the one or more aligners; the first exposure window is arranged in the catheter distal to the at least one of the one or more aligners; and the at least one of the one or more aligners is arranged in the catheter distal to the second exposure window. In some embodiments, the present disclosure relates to a system, further including a second catheter, wherein: the catheter is positionable through the second catheter; and the second catheter includes at least one of the one or more aligners. In some embodiments, the present disclosure relates to a system, further including one or more working channels to deliver a working volume in a direction of the first exposure window and/or the second exposure window to facilitate laceration, evacuation of debris, and visualization. In some embodiments, the present disclosure relates to a system, further including a second catheter, wherein: the catheter is positionable through the second catheter; and the second catheter includes an imaging system for visualization of tissue which is in contact with the one or more lacerators at the first exposure window and/or the second exposure window. In some embodiments, the present disclosure relates to a system, wherein the one or more aligners include at least one of: an expandable balloon; one or more hinged arms rotatably coupled to the catheter; or a deformable segment at a distal end of the catheter.

The present disclosure relates to a catheter for lacerating a tissue, including: one or more lacerators configured to lacerate the tissue at a first exposure window and a second exposure window; and one or more aligners, wherein the one or more aligners are deployable and configured to, when deployed, promote contact between the one or more lacerators and the tissue at the first exposure window and/or the second exposure window.

In some embodiments, the present disclosure relates to a catheter, wherein: the first exposure window is arranged in the catheter to enable a lacerator of the one or more lacerators to pierce the tissue to create an opening in the tissue; and a first aligner of the one or more aligners is arranged to, when deployed, promote contact between the tissue and the second exposure window when at least a part of the catheter is disposed in the opening of the tissue. In some embodiments, the present disclosure relates to a catheter, wherein: a second aligner of the one or more aligners is arranged to, when deployed, promote positioning of the catheter with respect to the tissue for piercing of the tissue by the lacerator.

The present disclosure relates to a method, including: advancing a catheter toward a tissue; piercing the tissue at a first exposure window of the catheter to generate an opening in the tissue; advancing at least a portion of the catheter through the opening in the tissue; deploying one or more aligners to promote contact between a lacerator and the tissue at a second exposure window of the catheter; slicing the tissue at the second exposure window of the catheter.

In some embodiments, the present disclosure relates to a method, wherein the method further includes: deploying a first aligner of the one or more aligners to promote positioning of the catheter with respect to the tissue for piercing of the tissue by the lacerator; and deploying a second aligner of the one or more aligners to promote contact between the tissue and lacerator at the second exposure window when at least a part of the catheter is disposed in the opening of the tissue. In some embodiments, the present disclosure relates to a method, wherein the first aligner is positioned on an opposite side of the tissue from the second aligner, when the second aligner is deployed.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 illustrates an example transcatheter surgery system 100 for transcatheter surgery in accordance with one or more embodiments.

FIG. 2 illustrates example catheter tip designs for a transcatheter surgery system 100 for transcatheter surgery in accordance with one or more embodiments.

FIG. 3 illustrates irrigation and aspiration features incorporated into a catheter tip of transcatheter surgery system 100 for transcatheter surgery in accordance with one or more embodiments.

FIG. 4 illustrates an example catheter, including one or more aligners, for transcatheter surgery systems in accordance with one or more embodiments.

FIG. 5 illustrates an example catheter, including one or more aligners, for transcatheter surgery systems in accordance with one or more embodiments.

FIG. 6 illustrates an example catheter, including one or more aligners, for transcatheter surgery systems in accordance with one or more embodiments.

FIG. 7 illustrates an example catheter, including one or more hinged arms as an aligner, for transcatheter surgery systems in accordance with one or more embodiments.

FIGS. 8A-8C illustrate an example method of performing transcatheter surgery with the catheter of FIG. 7 in accordance with one or more embodiments.

FIG. 9 illustrates an example catheter, including one or deformable portions as an aligner, for transcatheter surgery systems in accordance with one or more embodiments.

FIG. 10 illustrates an example method of performing transcatheter surgery with the catheter of FIG. 9 in accordance with one or more embodiments.

FIG. 11 illustrates an example handle system for a catheter for transcatheter surgery systems in accordance with one or more embodiments.

FIGS. 12A and 12B illustrate examples of catheters, including one or more mechanical mechanisms for deploying an aligner, for transcatheter surgery systems in accordance with one or more embodiments.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The present disclosure describes systems and methods of transcatheter surgery. The following embodiments provide technical solutions and technical improvements that overcome technical problems, drawbacks and/or deficiencies in the technical fields involving electro surgery and other transcatheter heart surgery technologies. As explained in more detail, below, technical solutions and technical improvements herein include aspects of improved calcification cutting, reducing invasiveness of surgeries and direct visualization of the region being operated on. Based on such technical features, further technical benefits become available to users and operators of these systems and methods. Moreover, various practical applications of the disclosed technology are also described, which provide further practical benefits to users and operators that are also new and useful improvements in the art.

Transcatheter tissue cutting is typically performed using transcatheter electrosurgery in which radiofrequency energy is passed through guidewires to cut or penetrate tissue inside the beating heart or inside blood vessels. To concentrate the energy at a specific location in the tissue, the guidewire is insulated except in the region of cutting and the blood is displaced with a nonconductive sterile solution such as dextrose.

Example valvular applications of transcatheter electrosurgery include the BASILICA (bioprosthetic or native aortic scallop intentional laceration to prevent iatrogenic coronary artery obstruction) technique, the LAMPOON (laceration of the anterior mitral leaflet to prevent left ventricular outflow obstruction) technique and the ELASTA-Clip (electrosurgical laceration and stabilization of MitraClip) technique.

However, there may be disadvantages to traditional transcatheter electrosurgical cutting. Three examples of these disadvantages are: (1) inability to cut through calcifications, (2) cumbersome procedures, and (3) inability to directly visualize the tissue.

The first disadvantage is that, while electrosurgery is effective at cutting through tissue, the technique may not be effective in cutting through the calcifications that are common in native and bioprosthetic valve leaflets.

The second limitation is that the procedure is cumbersome. To slice a leaflet using this conventional approach, a catheter loop must be formed through the leaflet. This involves first using electrosurgery to cut a hole through the leaflet. Then a second catheter must be introduced to snare the end of the first catheter that is protruding through the leaflet so that it can be pulled out of the patient. A V-shaped section of the first catheter over which the insulation has been removed must be pulled through the vasculature until it is aligned with the hole in the leaflet. Then, both ends of the first catheter must be pulled while electrifying the catheter to cut through the leaflet.

A third limitation is that, during cutting, the tissue is not being directly visualized since only ultrasound and/or fluoroscopy are available.

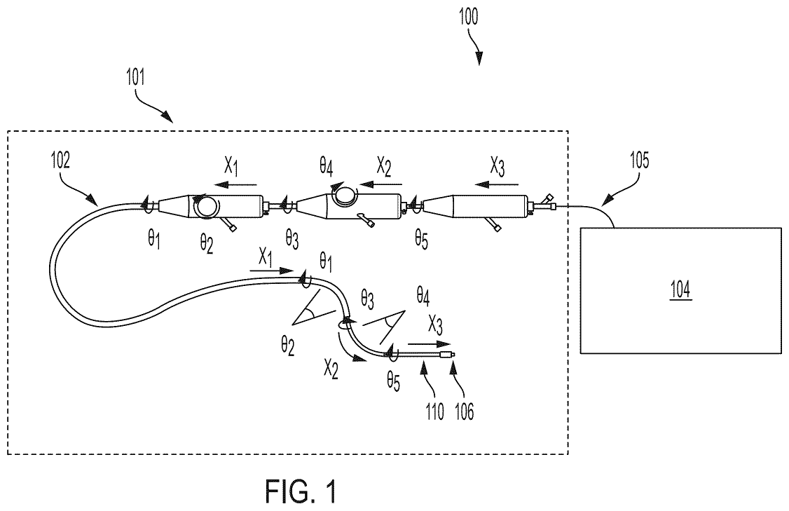

FIG. 1 illustrates an example transcatheter surgery system 100 for transcatheter surgery in accordance with one or more embodiments.

In some embodiments, transcatheter surgery may address or solve one or more of the above three disadvantages. In some embodiments, where the transcatheter surgery system 100 is a transcatheter laser surgery system, the use of lasers enables cutting of calcifications. Lasers are effective at cutting both tissue and mineralized deposits found in the body. For example, laser lithotripsy using Holmium:YAG and Thulium fiber lasers may prove highly effective technique for breaking up renal calculi, among other mineralized deposits and calcifications.

Embodiments described herein relate to lacerating tissue. The “tissue” described herein may be any desirable tissue. For instance the tissue may be native tissue, such as tissue grown or created by natural tissue growth of a subject animal (e.g., a human or non-human animal). Such tissue may be valve tissue, a tissue leaflet of a valve, cardiac tissue, or other tissue. As another example, the tissue may be non-native tissue, such as tissue grown by another animal (e.g., another human or non-human animal) and positioned within the subject animal, synthetic tissue positioned within the subject animal (e.g., a bioprosthetic valve leaflet), or other tissue. The tissue may be potentially calcified tissue.

In some embodiments, transcatheter procedures may be made less cumbersome through the design of one or more catheters used in the transcatheter surgery system 100. In some embodiments, transcatheter procedures may be made less cumbersome through the use of laser surgery. In some embodiments, the transcatheter surgery system 100 may include an energy source 104 couplable to at least one of one or more lacerators 105 and configured to be activated to transmit energy to the desired tissue. In some embodiments, the energy transmitted from the energy source 104 for transcatheter laser surgery, inside the heart for instance, may be delivered through the one or more lacerators 105 that may pass through a first catheter 110, or delivery tube, to the desired tissue. In some embodiments, the first catheter 110 may pass through a second catheter 101 having an outer sheath 102. In some embodiments, the second catheter 101 and outer sheath 102 may be steerable and/or rotatable. In some embodiments, the second catheter 101, and particularly the outer sheath 102 of the second catheter 101, may be used to deliver one or more elements, including the first catheter 110, the one or more lacerators 105 within the first catheter 110, working fluid, and/or a working volume, to a desired tissue. In some embodiments, by flowing saline or another fluid through the second catheter 101 around a distal end of the outer sheath 102, around a distal end of the first catheter 110, and/or around a distal end 106 of the one or more lacerators 105, blood can be evacuated from the contact area between the lacerator 105 and the tissue. The first catheter 110 may include stainless steel, nickel titanium, or any other desirable material. In some embodiments, as discussed in further detail below the first catheter 110 may include an inner tube and an intermediary sheath. In some embodiments, embolic filters may also be used to catch any debris that is generated. As used herein, the term “distal” refers to the direction away from a handle of the second catheter 101 (or first catheter 110), from which a user may manipulate the second catheter 101 (or first catheter 110). As used herein, the term “proximal” refers to the direction towards the handle of the second catheter 101 (or the first catheter 110).

The second and third limitations of traditional transcatheter electrosurgery described above are related. Standard catheter and imaging technology are accurate enough to position a catheter tip on a leaflet and burn a hole through that spot. It is not possible with standard catheter and imaging technology, however, to cut along an arbitrary line or curve through the tissue. Standard catheter and imaging technology only enables a wire loop through a hole in the tissue and then to create a cutting line in the direction that the loop can be pulled.

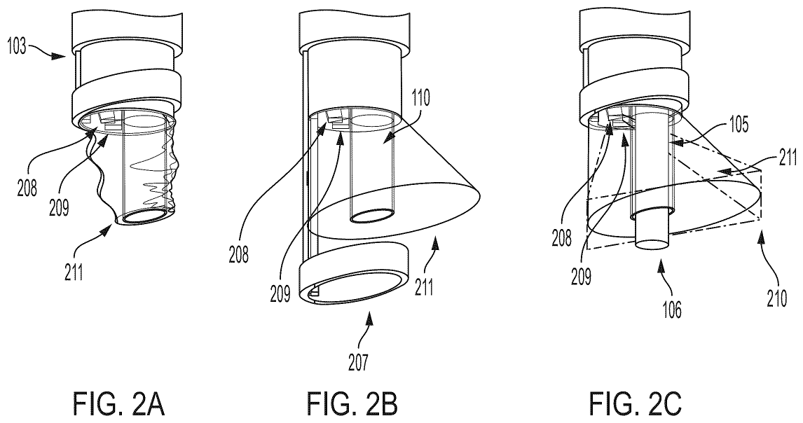

Referring to FIGS. 1 and 2, in some embodiments, the second catheter 101 may be a balloon catheter or any other suitable catheter type. For example, in some embodiments, the second catheter 101 may be a balloon catheter. In some embodiments, the second catheter 101 may include the outer sheath 102 and a balloon 211, which may be expanded, formed about the outer sheath 102 in sealed relation thereto. In some embodiments, the second catheter 101 includes an opening at its distal end that may be directed towards tissue of a patient and allow one or more devices or elements, such as the first catheter 110, to be passed therethrough towards the tissue of the patient. The opening at the distal end of the second catheter 101 may be at the distal end of the outer sheath 102. In some embodiments, the tissue may include potentially calcified tissue such as cardiac tissue including valvular tissue (e.g., leaflet, annulus, chordae, etc.) or arterial tissue, or other cardiac tissue.

In some embodiments, the balloon 211 may be located at the distal end of the second catheter 101 proximal to the tissue to be lacerated and in sealed relation to the outer sheath 102. By being located at the distal end of the second catheter 101 and located proximal to the tissue to be lacerated, the balloon 211 may be positioned and arranged with respect to the second catheter 101 and to the tissue to be lacerated such that, when the balloon 211 is inflated, at least a portion of the balloon 211 contacts the tissue. In some embodiments, fluid may be provided through the outer sheath 102 to the balloon 211 to inflate the balloon 211.

In some embodiments, the first catheter 110, including the one or more lacerators 105, may be advanced through the outer sheath 102 of the second catheter 101 such that the distal end of the first catheter 110 and the distal end 106 of at least one of the one or more lacerators 105 move towards or through the distal end of the second catheter 101. In some embodiments, the one or more lacerators 105 may be advanced through the first catheter 110 and outer sheath 102 of the second catheter 101 such that the distal end 106 of at least one of the one or more lacerators 105 moves towards or through the distal end of the first catheter 110 and the distal end of the second catheter 101. In some embodiments, the proximal ends of the one or more lacerators 105 may be coupled to the energy source 104. In some embodiments, the energy source 104 may be controllably activated to transmit energy through the one or more lacerators 105, and out the distal end 106 (or other portion) of the one or more lacerators 105, for instance, towards the tissue, thus irradiating the tissue to lacerate the tissue. Lacerate, as described herein, may include piercing or puncturing a hole through the tissue of interest. Lacerate, as described herein, may also include slicing or cutting a length of the tissue of interest.

In some embodiments, at least one of the one or more lacerators 105 includes an optical fiber, and the energy source 104 includes a laser energy source to transmit laser energy through the optical fiber. The distal end 106 of the optical fiber may include a fiber tip. In some embodiments, at least one of the one or more lacerators 105 includes an electrosurgical wire, and the energy source 104 includes an electrosurgical energy source, such as an RF energy source, to transmit electrosurgical energy through the electrosurgical wire. The distal end 106 of the electrosurgical wire may include an electrode. In electrosurgical embodiments, the system may be monopolar or bipolar. In monopolar systems, the electrosurgical wire may have one uninsulated electrode on it (toward its distal end 106, for instance) and the patient would have a second electrode attached to or otherwise contacting the patient in another location, such as attached via patches to the patient's back or buttocks, for example. In bipolar systems, there may be two electrodes spaced a small distance apart and coupled to one or more electrosurgical wires (at or near a distal end 106 of the one or more electrosurgical wires, for instance) and the energy would flow through the tissue between the two electrodes. Therefore, in some embodiments the one or more lacerators 105 is one or more electrodes, and the energy source is an electrosurgical energy source configured to transmit electrosurgical energy to the tissue to be lacerated, the transmitted electrosurgical energy passing from or between the one or more electrodes through the tissue. In some embodiments, at least one of the one or more lacerators 105 may be suitable to transmit any other desirable energy source, including thermal energy and/or cryogenic energy, for instance, and the energy source 104 may be suitable to transmit the any other desirable energy to the one or more lacerators 105. In some embodiments, at least one of the one or more lacerators 105 may be a mechanical lacerator, such as a sharpened blade, and may or may not be coupled to the energy source 104.

In some embodiments, transcatheter surgery according to the present disclosure may include a steerable catheter system. The one or more lacerators 105 may be incorporated into the steerable catheter system, thus combining the energy delivery mechanism with the steerable catheter system. In some embodiments, the combination enables precise positioning and motion of the one or more lacerators 105, for instance the distal end 106 of the one or more lacerators 105, with respect to the desired tissue to be lacerated. This functionality can enable lacerating of a valve leaflet, for example, without the steps of forming a wire loop, thus minimizing the steps needed to effectively complete the surgery and reduce invasiveness. Furthermore, the direction of lacerating is not limited to the direction defined by pulling of the loop. For example, only base-to-tip leaflet laceration is possible with the typical transcatheter electrosurgery technique. It may be desirable to lacerate a different pattern in a leaflet or to completely remove it, which transcatheter laser laceration makes possible.

FIG. 2 illustrates example catheter tip designs for the transcatheter surgery system 100 for transcatheter surgery in accordance with one or more embodiments.

In some embodiments, to further improve the safety and effectiveness of tissue laceration, the transcatheter surgery system 100 may include one or more working channels to deliver a working volume at a catheter tip 103 of the second catheter 101, and in some embodiments a distal end of the first catheter 110, to facilitate laceration, evacuation of debris, and visualization. The catheter tip 103 may be positioned at the distal end of the second catheter 101. The catheter tip 103, through which the first catheter 110 and one or more lacerators 105 may be delivered can be constructed to include a working volume incorporated into the catheter tip 103 that may surround the one or more lacerators 105. In some embodiments, the working volume may act to seal off the laceration zone from the surrounding blood. This volume can be connected to one or more working channels in the second catheter 101 for irrigation and aspiration. For example, irrigation of a sterile solution such as saline to force out the blood and to enhance cutting. Aspiration may be used to evacuate cutting debris. In some embodiments, by sealing off the laceration zone from surrounding blood, which is opaque, the working volume may enhance imaging of the laceration zone during tissue laceration, which is discussed in greater detail below.

In some embodiments, the catheter tip 103 may include an extending member that encloses the working volume on a back face of a tissue layer of the tissue being cut.

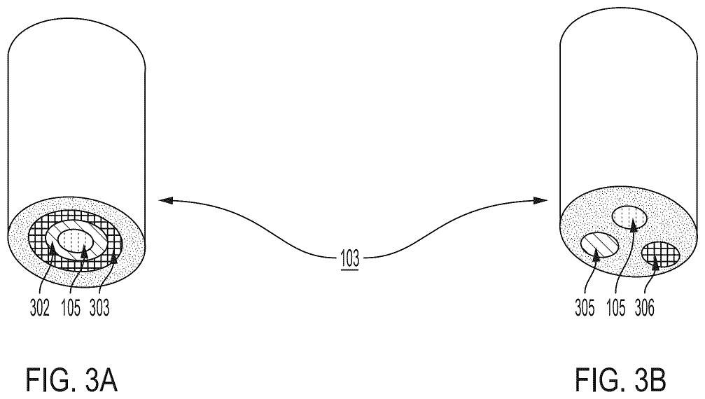

In some embodiments, catheter tip 103 can be designed in many ways. In some embodiments, first catheter 110 may include a larger-diameter lumen for the one or more lacerators 105, where the larger-diameter lumen may allow saline, dextrose, or other fluid to flow out of the first catheter 110, at for instance, the distal end of the first catheter 110. Alternate designs of the catheter tip 103 include concentric working channels providing both irrigation and aspiration (see FIG. 3). Furthermore, catheter tip 103 can include endoscopic imaging enabling visualization during tissue laceration, e.g., using a camera 208 and a light emitting diode (LED) 209 for imaging and illumination, respectively. In some embodiments, an optical window 210 for visualization may be clear solid or it may be balloon 211 as shown here. (A) of FIG. 2 shows the balloon 211 is deflated for navigation through vasculature. (B) of FIG. 2 shows the balloon 211 is inflated to create larger diameter contact region with tissue and a grasping finger 207 is used to hold tissue against optical window 210 during cutting. Tissue grasping finger 207 may be solid cup shape (not shown) in order to form enclosed volume around tissue being cut. (C) of FIG. 2 shows the optical window 210 is shown with grasping finger 207 retracted. The tissue grasping finger 207 may be an aligner, discussed in further detail below, for promoting contact between a tissue and a lacerator 105 for piercing the tissue. In some embodiments, the lacerator 105 may be advanced through the tissue and the grasping finger such that the grasping finger 207 acts as an aligner during pushing and/or pulling laceration procedures, as discussed in further detail below. In some embodiments, the balloon 211 may center, or otherwise precisely position the first catheter 110 and/or the one or more lacerators 105 in the optical window 210. In some embodiments, the position and orientation of the one or more lacerators 105 may be fixed with respect to the working volume or the one or more lacerators 105 may be moveable with respect to the working volume.

In some embodiments, to further improve the safety and effectiveness of leaflet cutting, the catheter tip 103 through which the first catheter 110 and/or one or more lacerators 105 are delivered can be constructed to include an optical imaging device/system including, e.g., the camera 208 and/or LED 209 incorporated with the catheter tip 103 so that laceration may be directly visualized. In some embodiments, using a clear fluid, such as saline, to fill the working volume may facilitate imaging while also enhancing laceration.

In some embodiments, to further improve the safety and effectiveness of tissue laceration, the catheter tip 103 through which the first catheter 110 and/or one or more lacerators 105 are delivered can be constructed to include a catheter tip 103 having an extending member with a capability to grasp a leaflet or other tissue, e.g., using a grasping feature or grasping mechanism such as the tissue grasping finger 207 of FIG. 2. In some embodiments, the catheter tip 103 with the grasping capability may be used in order to control motion of the leaflet or other tissue with respect to the catheter during laceration and/or to control the motion of the catheter with respect to the leaflet or other tissue during laceration. In some embodiments, the catheter tip 103 with the grasping capability may also be used to separate the leaflet or other tissue from surrounding tissue to enable cutting the full thickness of the tissue without damaging distal surrounding structures.

In some embodiments, grasping feature may also be used to completely enclose the working volume (including, e.g., the catheter tip 103, surrounding leaflet tissue, the grasping feature, etc.) to contain fluids and debris associated with cutting.

In some embodiments, the transcatheter surgery system 100 may include an embolic filter used to catch laceration debris escaping into the blood. In some embodiments, the embolic filter may be incorporated into the catheter tip 103, the outer sheath 102, or may be a separate component.

FIG. 3 illustrates irrigation and aspiration features incorporated into a catheter tip 103 of transcatheter surgery system 100 for transcatheter surgery in accordance with one or more embodiments.

In some embodiments, the working channels of the catheter tip 103 may provide and remove working fluid of a working volume, including, e.g., for irrigation and aspiration to optimize laceration and removal of debris. In some embodiments, (A) and (B) of FIG. 3 show two examples of working channels for irrigation and aspiration.

In some embodiments, (A) of FIG. 3 depicts concentric channels of working channels, with a lacerator 105, and in some embodiments the first catheter 110 and the lacerator 105, enveloped by an inner lumen 302 for irrigation and an outer lumen 303 for aspiration.

In some embodiments, (A) of FIG. 3 depicts alternate channels of working channels, with lacerator 105, and in some embodiments the first catheter 110 including the lacerator 105, in a first lumen separated from a second lumen 305 for irrigation and a third lumen 306 for aspiration. In some embodiments, in the alternate channel arrangement, each lumen 305/306 and the lumen for the lacerator 105, and in some embodiments the first catheter 110, are separate openings in the catheter tip 103.

While embodiments have been described herein where the aspiration, irrigation, and imaging components of the system 100 are positioned through outer sheath and tip 103 of the second catheter 101, it should be appreciated that this is a non-limiting example. For instance, the first catheter 110 may include any or all of the balloon 211, grasping finger 207, camera 208, LED 209, irrigation lumen 302/305, and/or aspiration lumen 303/306, such that any or all of the components extend through and are deployed from the first catheter 110.

In some embodiments, the transcatheter surgery system 100 may include one or more aligners to promote contact between the one or more lacerators 105 and the tissue to be lacerated. In some embodiments, the first catheter 110 may include at least one of the one or more aligners. In some embodiments, the second catheter 101 may include at least one of the one or more aligners. In some embodiments, the one or more aligners are deployable, such that the one or more aligners promote contact between the one or more lacerators 105 and the tissue when the one or more aligners are deployed. In some embodiments, when deployed, the one or more lacerators 105 may expand outwardly from the first catheter 110 and/or the second catheter 101.

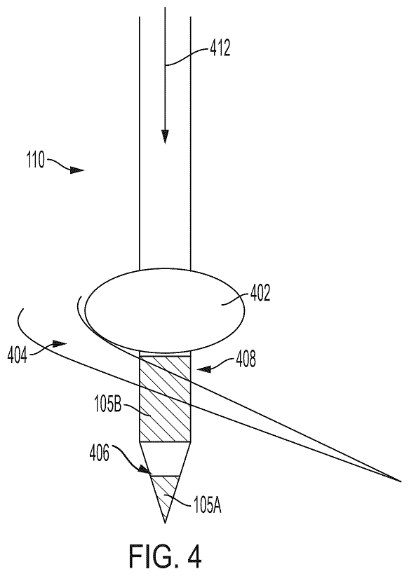

Referring now to FIG. 4, the first catheter 110 is depicted with an aligner 402. While not shown, it should be appreciated that the first catheter 110 may be at least partially positioned in the second catheter 101 described in any of the above embodiments. Additionally, the first catheter 110 may include an inner tube and intermediary sheath, as discussed further below. The aligner 402 may be deployable coupled to the first catheter 110. The aligner 402 may be an expandable balloon, a hinged arm, a deformable portion of the first catheter 110, or any other suitable device or mechanism for maintaining contact between the lacerator 105 and a tissue 404. The tissue 404 is depicted as a cross-sectional view of a leaflet, but it should be appreciated that any other desirable tissue may be lacerated. One or more control mechanisms pay pass through the first catheter 110 proximally such that a user may selectively deploy the aligner 402. For instance, in some embodiments a fluid channel may pass through the first catheter 110 to allow for inflation or expansion of a balloon aligner 402. In some embodiments, a trigger at a proximal end of the first catheter 110 may allow a user to selectively deploy and expand a hinged arm, for instance.

In some embodiments, a first exposure window 406 of the lacerator 105 and a second exposure window 408 of the lacerator 105 may allow for contact between the lacerator 105 and the tissue 404. In some embodiments, the first exposure window 406 and the second exposure window 408 are contiguous. In some embodiments, the first exposure window 406 and the second exposure window 408 are arranged in the first catheter 110 as openings or partial openings in an outer surface of the first catheter 110. For instance, in some embodiments, the first exposure window 406 may be arranged as an axial opening at the distal-most end of the first catheter 110, allowing the lacerator 105 to be passed axially out of the first catheter 110 in the distal direction. In some embodiments, the second exposure window 408 may be formed as a slot in a radially outer wall of the first catheter 110, allowing the lacerator 105 to contact the tissue 404 in both an axial and lateral direction at the second exposure window 408, as discussed in more detail below. In some embodiments, the first exposure window 406 is arranged in the catheter 110 distal to the second exposure window 408, and the second exposure window 408 is arranged in the catheter 110 distal to the aligner 402.

In some embodiments, the lacerator 105 may be a single lacerator movable through the first catheter 110 such that the lacerator 105 is selectively exposable at the first exposure window 406 and/or the second exposure window 408 depending on the distal advancement of the lacerator 105 in the first catheter 110. In some embodiments, the first catheter 110 may include two lacerators 105A and 105B. In some embodiments, the first lacerator 105A may be exposable through the first exposure window 406 to contact the tissue 404, and the second lacerator 105B may be exposable through the second exposure window 408 to contact the tissue 404. In some embodiments, the first lacerator 105A and the second lacerator 105B may be of the same type. For instance, the first lacerator 105A and the second lacerator 105B may each be optical fibers to transmit laser energy, or the first lacerator 105A and the second lacerator 105B may each be electrosurgical wires (or electrodes) to transmit electrosurgical energy. In some embodiments, the first lacerator 105A and the second lacerator 105B may be of different types. For instance, the first lacerator 105A may be an optical fiber, and the second lacerator 105B may be an electrosurgical wire (or electrode), or vice versa.

In some embodiments, the lacerator 105, or the first lacerator 105A, at the first exposure window 406, may pierce the tissue 404 to create an opening in the tissue 404. In some embodiments, the lacerator 105, or the second lacerator 105B, at the second exposure window 408, may slice the tissue 404. In some embodiments, with the first catheter 110 at least partially positioned through the hole in the tissue 404 created by the piercing of the tissue 404, as shown in FIG. 4, the first catheter 110 and/or the lacerator 105 (or, in embodiments the second lacerator 105B) may be axially oscillated and moved laterally to slice the tissue 404. In some embodiments, the aligner 402 may be deployed after the first catheter 110 is at least partially positioned through the hole in the tissue 404 created by the piercing to maintain sufficient contact between the lacerator 105 (or in embodiments, the second lacerator 105B) and the tissue 404 at the second exposure window 408 to promote desired slicing of the tissue 404.

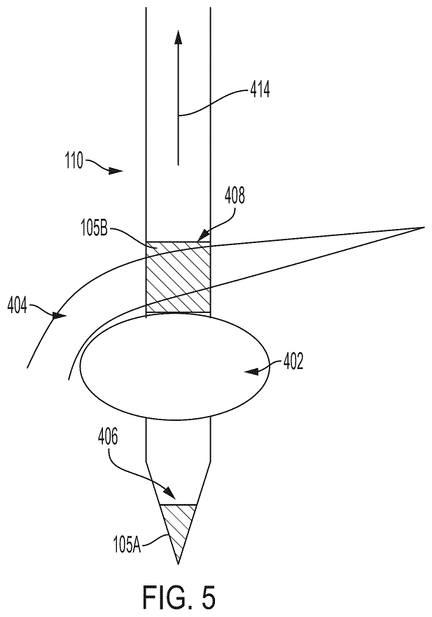

Referring now to FIG. 5, another embodiment of a first catheter 110 is depicted. In FIG. 5, like numerals are used to depict the same or similar features as discussed with respect to FIG. 4. The first catheter 110 depicted in FIG. 5 may resemble the first catheter 110 depicted in FIG. 4 in form and function, except as noted herein. Particularly, with reference to FIG. 5, the first exposure window 406 is arranged in the catheter 110 distal to the aligner 402, and the aligner 402 is arranged in the catheter 110 distal to the second exposure window 408. As discussed with reference to FIG. 4, the aligner 402 may be deployed after the first catheter 110 is at least partially positioned through the hole in the tissue 404 created by the piercing to maintain sufficient contact between the lacerator 105 (or in embodiments, the second lacerator 105B) and the tissue 404 at the second exposure window 408 to promote desired slicing of the tissue 404.

With reference to FIGS. 4 and 5, the particular design of the first catheter 110 may be selected depending on the particular tissue 404 to be lacerated, the particular anatomy of a patient, the angle or direction of approach towards the desired tissue to be lacerated, etc. In some embodiments, the first catheter 110 of FIG. 4 may be particularly advantageous when lacerating a tissue, such as a leaflet, by pushing (i.e. when the catheter 110 is advanced in the direction 412 depicted in FIG. 4 to slice the tissue). In some embodiments, the first catheter 110 of FIG. 5 may be particularly advantageous when lacerating a tissue, such as a leaflet, by pulling (i.e. when the catheter 110 is advanced in the direction 414 depicted in FIG. 5 to slice the tissue).

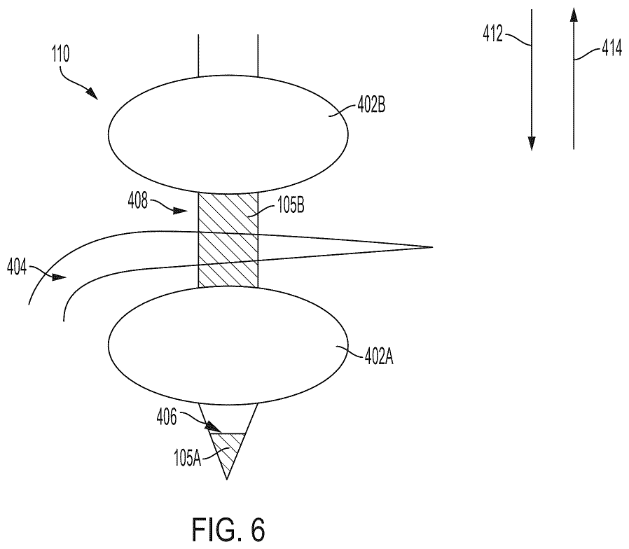

With reference to FIG. 6, another embodiment of a first catheter 110 is depicted. In FIG. 6, like numerals are used to depict the same or similar features as discussed with respect to FIGS. 4 and 5. The first catheter 110 depicted in FIG. 6 may resemble the first catheters 110 depicted in FIGS. 4 and 5 in form and function, except as noted herein. Referring to FIG. 6, the first catheter 110 includes a first aligner 402A and a second aligner 402B. The first exposure window 406 is arranged in the catheter 110 distal to the first aligner 402A, the first aligner 402A is arranged in the catheter 110 distal to the second exposure window 408, and the second exposure window 408 is arranged in the catheter 110 distal to the second aligner 402B. The first aligner 402A and the second aligner 402B may be of the same or different types (e.g., balloons, hinged arms, etc.). The first aligner 402A and the second aligner 402B may be independently deployable, or expandable. In some embodiments, when proximal the tissue 404, the first aligner 402A may be deployed to promote contact between the lacerator 105 (or in some embodiments the first lacerator 105A) and the tissue 404 at the first exposure window 406 to pierce the tissue 404. In some embodiments, the lacerator 105 may pierce the tissue 404 at the first exposure window 406 without deployment of the first aligner 402A. In embodiments when deployed to pierce, the first aligner 402A may then be collapsed or deflated such that a portion of the first catheter 110, including the first aligner 402A may be advanced through the opening pierced in the tissue 404. For laceration by pushing (i.e. in the direction of arrow 412) the second aligner 402B may then be deployed or expanded to promote contact between the lacerator 105 (or in some embodiments the second lacerator 105B) and the tissue 404 at the second exposure window 408 to slice the tissue 404. Therefore, the first aligner 402A is positioned on an opposite side of the tissue 404 from the second aligner 402B when the second aligner 402B is deployed or for laceration by pulling (i.e. in the direction of arrow 414), after the tissue 404 is pierced and the catheter 110 advanced through the opening in the tissue 404, the first aligner 402A can be deployed or expanded to promote contact between the lacerator 105 (or in some embodiments the second lacerator 105B) and the tissue 404 at the second exposure window 408 to slice the tissue 404.

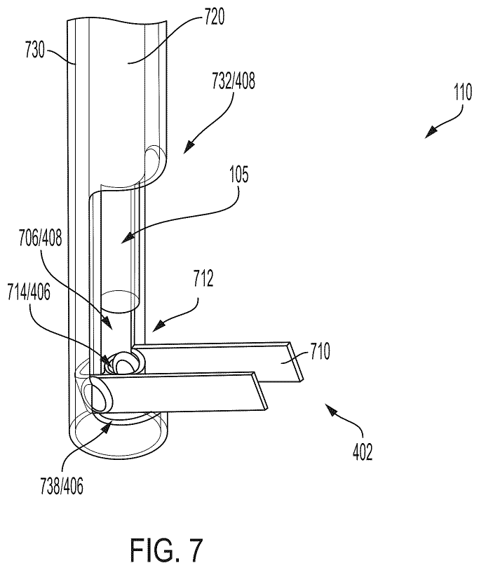

Referring now to FIG. 7, a perspective view of the first catheter 110 including one or more hinged arms 710 as an aligner 402 is depicted. The first catheter 110 is depicted as including the inner tube 720 and the intermediary sheath 730, which surrounds the inner tube 720. The inner tube 720 and the intermediary sheath 730 may be movable with respect to each other. The inner tube 720 of the first catheter 110 may include a notch 706 to define at least a portion of the second exposure window 408. The intermediary sheath 730 of the first catheter 110 may include a notch 732 that may overlap with the notch 706 to define at least a portion of the second exposure window 408. The lacerator 105 may be exposed through the overlapped notches 706, 732. An opening 714 at the distal most end of the inner tube 720 of the first catheter 110 may define at least a portion of the first exposure window 406. An opening 738 at the distal most end of the intermediary sheath 730 may overlap with the opening 714 and at least partially define the first exposure window 406. In some embodiments, the one or more hinged arms 710 may be rotatably coupled to the inner tube 720 of the first catheter 110 by means of a double pin joint 712. The one or more hinged arms 710 may be rotated away from the longitudinal axis of the first catheter 110 to be deployed or expanded outward. The one or more hinged arms 710 may be rotated inwardly toward the longitudinal axis of the first catheter 110 to be collapsed. The double pin joint 712 may allow the lacerator 105 to pass therethrough without ablating the double pin joint 712 and/or the one or more hinged arms 710. For example, and as discussed above, the lacerator 105 may be distally advanced through the openings 714, 738 to puncture a tissue. Following puncturing, the lacerator 105 may be retracted proximally in the first catheter 110 to allow tissue to collapse into the notches 706, 732. Then the lacerator may be advanced distally in the first catheter 110 to slice the tissue that collapsed into the notches 706, 732. This retraction and advancement of the lacerator 105 may be repeated to slice the tissue along a desired path. For instance, in some embodiments, the lacerator 105 may be oscillated axially within the notches 706, 732 as the first catheter 110 is moved laterally across a leaflet in the desired cutting direction. The one or more hinged arms 710 as depicted may particularly enable retrograde laceration of a leaflet.

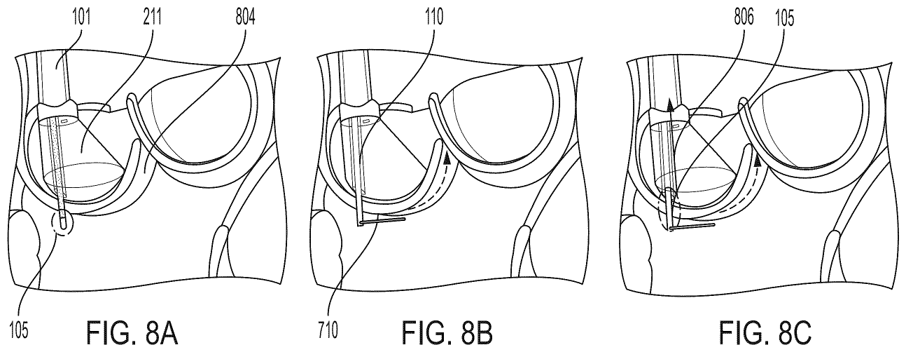

Referring now to FIGS. 8A-8C, an example method of using the first catheter 110 of FIG. 7 is depicted. As discussed with reference to FIG. 2, a balloon 211 may be deployed from the outer catheter 101 and an optical window 210 (FIG. 2) can be generated. In addition to having the aforementioned benefits discussed with references to FIG. 2, the balloon 211 may also assist in maintaining contact between the lacerator 105 and a tissue 804, such as maintaining contact at a desired location. For example, the balloon 211 may, when expanded, have an outer shape that conforms to a shape of at least a portion of the tissue 804, where the portion corresponds to a desired location of the catheter 101. By expanding the balloon 101 and then contacting the tissue 804 with the balloon 211, the balloon 211 may move into a desired position where the outer shape aligns with the portion and, by moving, the balloon 211 may cause the catheter 101 to move into a desired position. It should be appreciated, though, that the balloon 211, while depicted, is not required for use with the first catheter 110. In FIG. 8A, once the catheter 101 is in a desired position (e.g., as a result of the balloon 211), the lacerator 105 may be advanced distally though the openings 714, 738 (FIG. 7) of the first catheter 110 to puncture the tissue 804. Following this, the first catheter 110 may be advanced though the opening punctured in the tissue 804 with the one or more hinged arms 710 collapsed. After insertion through the opening in the tissue 804, the one or more hinged arms 710 may be deployed outwardly as shown to maintain contact between the lacerator 105 and the tissue 804 at the notches 706, 732 (FIG. 7). As shown in FIG. 8C, the first catheter may then be pulled in the direction of arrow 806 to lacerate the tissue 804. While the first catheter 110 discussed in FIG. 7 and FIGS. 8A-8C included the inner tube 720 and the intermediary sheath 730, it should be appreciated that in some embodiments the first catheter 110 may only be defined by the inner tube 720, and the intermediary sheath 730 may not be incorporated into the first catheter 110.

While the one or more hinged arms 710 have been particularly discussed with respect to FIG. 7 and FIGS. 8A-8C, it should be appreciated that the one or more hinged arms 710 are merely one example of an aligner 402. For instance, the arms of the one or more hinged arms 710 need not be coupled to the inner tube 110 with the double pin joint 712, as depicted. Other mechanisms may be used in place of the double pin joint 712 and/or the one or more hinged arms 710. Merely as an example, a slider crank may be used to deploy an aligner 402. In some embodiments, the lacerator 105 may be coupled to the aligner 402 and be at least a portion of a mechanism that transitions the aligner between collapsed and deployed configurations.

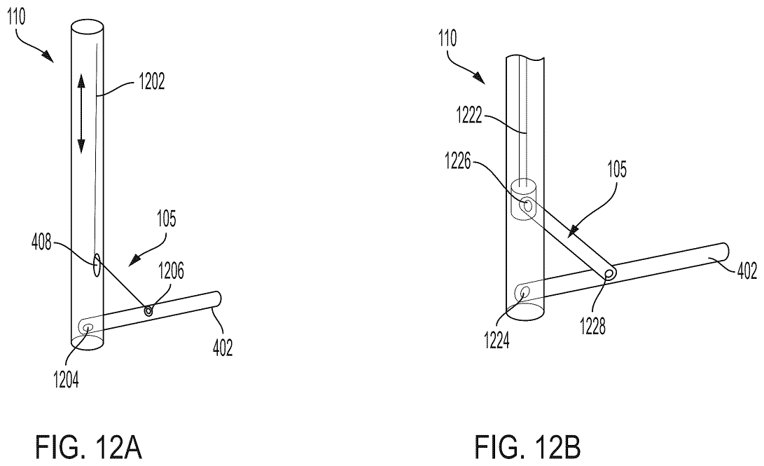

Referring now to FIG. 12A, an embodiment of the first catheter 110 is depicted. The aligner 402 may be coupled to the first catheter 110 at a joint 1204. The lacerator 105 may be coupled to a control wire 1202 that extends proximally through the first catheter 110. Distal advancement of the control wire 1202 may advance the lacerator 105 through the second exposure window 408 in the first catheter 110. A distal end 1206 of the lacerator 105 may be coupled to the aligner 402. Distal advancement of the control wire 1202 therefore simultaneously exposes the lacerator 105 through the second exposure window 408 and transitions the aligner 402 from a collapsed configuration to a deployed configuration by causing rotation of the aligner 402 about the joint 1204. Moving the control wire 1202 in the proximal direction may then withdraw the lacerator 105 such that the lacerator 105 is not exposed through the second exposure window 408 and the aligner 402 transitions from the deployed configuration to the collapsed configuration.

Referring now to FIG. 12B, an embodiment of the first catheter 110 is depicted. The aligner 402 may be coupled to the first catheter 110 at a joint 1224. The lacerator 105 may be coupled to slider 1222. Particularly, a proximal end of the lacerator 105 may be coupled to a distal end of the slider 1222 at a joint 1226. A distal end of the lacerator 105 may be coupled to the aligner 402 at a joint 1228. The joints 1224, 1226, and 1228 may be pin joints, for instance. Distal advancement of the slider 1222 may advance the lacerator 105 through the second exposure window 408 (FIG. 12A) in the first catheter 110. Due to the coupling of the lacerator 105 to the aligner 402, distal advancement of the slider 1222 may simultaneously expose the lacerator 105 through the second exposure window 408 (FIG. 12A) and transitions the aligner 402 from a collapsed configuration to a deployed configuration by causing rotation of the aligner 402 about the joint 1224. Moving the slider 1222 in the proximal direction may then withdraw the lacerator 105 such that the lacerator 105 is not exposed through the second exposure window 408 (FIG. 12A) and the aligner 402 transitions from the deployed configuration to the collapsed configuration.

In the embodiments, discussed with reference to FIGS. 12A and 12B, a mechanical mechanism transitions the aligner 402 between collapsed and deployed configurations. In some embodiments, the mechanical mechanism may be a hinge mechanism. The lacerator 105 may define at least a portion of the mechanical mechanism that transitions the aligner 402 between collapsed and deployed configurations. In the embodiments depicted in FIGS. 12A and 12B, it should be appreciated that a second lacerator (e.g. the first lacerator 105A depicted in FIGS. 4-6) may be employed for exposure through the first exposure window 406 (FIGS. 4-6). In some embodiments, the lacerator 105 discussed with respect to FIGS. 12A and 12B is an electrosurgical lacerator.

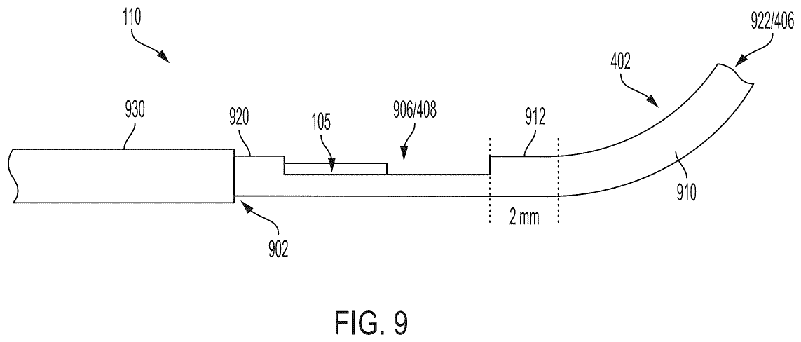

Referring now to FIG. 9, another embodiment of the first catheter 110 is depicted. The first catheter 110 may include an intermediary sheath 930 and an inner tube 920. The intermediary sheath 930 may include an opening 902 at its distal end through which the inner tube 920 may be advanced. The opening 902 may be in a distal end wall of the intermediary sheath 930. The inner tube 920 may at least partially be included in the cannula of the intermediary sheath 930. The inner tube 920 may include a notch 906, defining the second exposure window 408, in a sidewall of the inner tube 920, through which the lacerator 105, may be exposed. In some embodiments, a portion of the inner tube 920 defines the aligner 402. In some embodiments, a distal most portion of the inner tube 920 defines the aligner 402. In some embodiments, the aligner 402 may be coupled to or positioned on the inner tube 920 at a distal end of the inner tube. In some embodiments, the aligner 402 may be a deformable portion 910 of the inner tube 920. In some embodiments, the deformable portion 910 may be elastically deformable such that the deformable portion 910 is straight (in relation to the longitudinal axis of the notch 906, for instance) when the deformable portion 910 is positioned within the intermediary sheath 930. In some embodiments, when deformable portion 910 is distally advanced out of the intermediary sheath 930, the deformable portion 910 may deform, or deploy, into the curved shape as shown. In some embodiments, the inner tube 920 includes a straight segment 912 distal to the notch 906 and proximal to the deformable portion 910. The straight segment 912 may be greater than or equal to 1 mm in length. In some embodiments, the straight segment 912 is 2 mm in length. In some embodiments, the straight segment 912 ensures that the deformable portion 910 is not ablated as the lacerator 105 is moved distally in the notch 906. In some embodiments, the lacerator 105 may be oscillated axially within the inner tube 920 as the first catheter 110 is moved laterally across a leaflet in the desired cutting direction. An opening 922 may be formed in the most distal axial face of the inner tube 920 defining the first exposure window 406. When the deformable portion 910 is straight, the lacerator 105 may be advanced through the deformable portion 910 and the opening 922 to pierce a tissue. The deformable portion 910 may particularly enable antegrade laceration of a tissue.

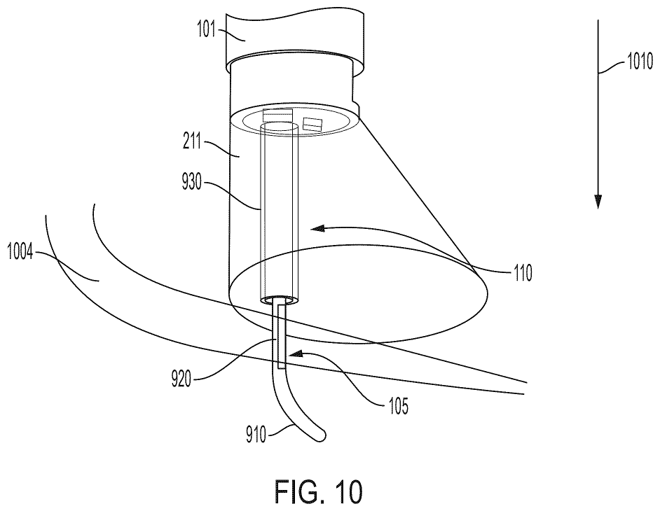

Referring now to FIG. 10, an example method of using the first catheter 110 of FIG. 9 is depicted. As discussed with reference to FIG. 2, a balloon 211 may be deployed from the outer catheter 101 and an optical window 210 (FIG. 2) can be generated. In addition to having the aforementioned benefits discussed with references to FIG. 2, the balloon 211 may also assist in maintaining contact between the lacerator 105 and a tissue 1004, as discussed. With the inner tube 920 positioned in the intermediary sheath 930, the lacerator may be advanced distally out of the opening 922 (FIG. 9) to pierce a hole in the tissue 1004. Following the piercing, the lacerator 105 may be retracted proximally a distance and the inner tube 920 may be advanced out of the intermediary sheath 930 and through the hole in the tissue 1004, allowing the deformable portion 910 to deploy and curve outwardly. The lacerator 105 may then be advanced into the notch 906 (FIG. 9) for slicing the tissue 1004. If the first catheter 110 is advanced distally in a pushing motion in the direction of the arrow 1010, the deployed deformable portion 910 assists the balloon 211 in maintaining contact between the tissue 1004 and the lacerator 105. The lacerator may be oscillated in the notch 906 (FIG. 9) as discussed in prior embodiments to slice the tissue 1004. While the balloon 211 is shown and described as being coupled to the second catheter 101 and functioning as an aligner, it should be appreciated that the balloon 211 may instead be coupled to the first catheter 110, positioned similarly to the aligner 402 depicted in FIG. 4.

It should be appreciated the embodiments of FIGS. 4-10 may be combined with any or all of the features discussed with respect to FIGS. 1-3, as desired. For instance, the first catheters 110 discussed may be deployed with the second catheter 101 that may include any or all of the balloon 211, the camera 208, the LED 209, the tissue grasping finger 207, the first lumen 305/302 for irrigation, and/or the second lumen 306/303 for aspiration. Moreover, it should be appreciated that one or more components of the second catheter 101, such as the balloon 211 and grasping finger 207 may be aligners that promote contact between the lacerator 105 and tissue to be lacerated. Similarly, it should be appreciated that any or all of the components discussed with respect to the second catheter 101 in FIGS. 1-3, such as the balloon 211, the camera 208, the LED 209, the tissue grasping finger 207, the first lumen 305/302 for irrigation, and/or the second lumen 306/303 for aspiration, may instead be incorporated into the first catheter 110. The embodiments discussed and depicted with respect to FIGS. 4-10 may offer several advantages. The first is that the tool may remain inserted through the leaflet traversal hole until laceration is complete. Second, the tool may assist in maintaining contact between the cardioscope balloon and the leaflet for visualization during cutting. Third, the tool may concentrate electrical current and/or laser energy (or other laceration energy) on the tissue in the desired cutting direction. Fourth, the tool may permit dextrose or saline solution infusion around the cutting mechanism.

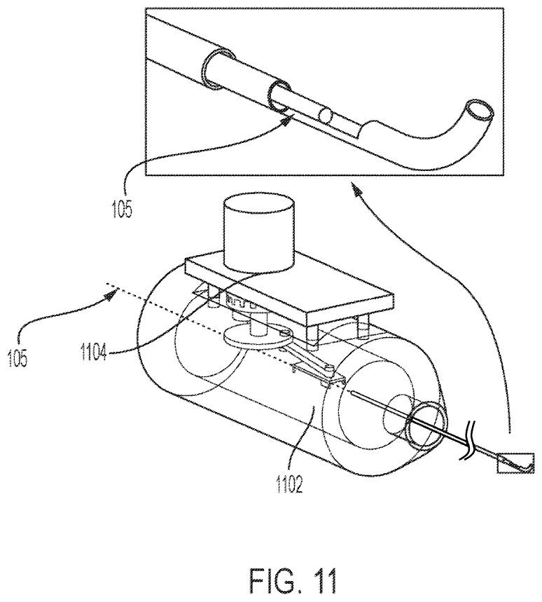

In any of the above-described embodiments, while the operator may manually control catheter motion during laceration, lacerator 105 (e.g. mechanical, optical fiber, or electrosurgical wire/electrode) oscillation may be motorized. For instance, with reference to FIG. 11, the transcatheter surgical tool may include a motorized handle. The lacerator 105 may pass through a valve at the proximal end of the handle and be fixed using a thumbscrew clamp to a motorized oscillating mechanism 1102. The motor unit 1104 will couple to the handle through a snap attach/release mechanism so that it can be bagged for sterility. Based on handle ergonomics and cost, a variety of handle drive mechanisms, including a slider crank, voice coil motor with spring return, or pneumatic actuation may be implemented. A port in the handle may allow pump-based saline or dextrose infusion to the cutting tool. Foot pedal control may enable a single operator to simultaneously activate the lacerator 105 (e.g. optical fiber or electrosurgical wire/electrode), motorized lacerator oscillation, and saline or dextrose infusion.

In some embodiments, the transcatheter surgery system(s) and method(s) is not limited to the specific advantages and procedures described herein and may include other catheter tip surgical procedures.

In some embodiments, control of the transcatheter surgery system(s) 100 and/or component(s) (e.g., the lacerator 105, catheter tip, grasping feature, working volume, steerable catheter system, optical imaging device, among other system(s) and/or component(s) or any combination thereof) may be effectuated with one or more control algorithms executed by one or more computing devices. In some embodiments, the one or more control algorithms may include software that when executed by the one or more computing devices, controls the transcatheter surgery system(s) and/or component(s) to perform operations of a procedure, e.g., either automatically, by manual input, or a combination thereof. In some embodiments, the software may be implemented as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

In some embodiments, the one or more computing devices may include computer engines. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the one or more computing devices may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

It should be appreciated that the above-described one or more control algorithms executed by one or more computing devices may be implemented to control transcatheter surgical systems of any desired cutting mechanism, including, for instance, laser-based cutting mechanisms and/or electrosurgical cutting mechanisms.

The aforementioned examples are, of course, illustrative and not restrictive.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1.-20. (canceled)

21. A catheter for lacerating a tissue, comprising:
one or more exposure windows;
one or more lacerators, wherein the one or more lacerators are configured to lacerate the tissue and are deployed at or from the one or more exposure windows; and
one or more aligners, wherein the one or more aligners are deployable and configured to, when deployed, promote contact between the one or more lacerators and the tissue.

22. The catheter of claim 21, wherein:
the one or more exposure windows comprise a first exposure window;
the first exposure window is arranged in the catheter to enable a lacerator of the one or more lacerators to pierce the tissue to create an opening in the tissue; and
a first aligner of the one or more aligners is arranged to, when deployed, promote contact between the tissue and a lacerator of the one or more lacerators when at least a part of the catheter is disposed in the opening of the tissue.

23. The catheter of claim 22, wherein:
the one or more exposure windows further comprise a second exposure window; and
the second exposure window is arranged in the catheter to enable the lacerator of the one or more lacerators to slice the tissue when the lacerator of the one or more lacerators is deployed at or from the second exposure window of the catheter.

24. The catheter of claim 21, wherein:
the one or more exposure windows comprise a first exposure window and a second exposure window; and
the one or more lacerators comprise:
a first lacerator advanceable through the catheter such that the first lacerator is exposable through the first exposure window; and
a second lacerator advanceable through the catheter such that the second lacerator is exposable through the second exposure window.

25. The catheter of claim 21, wherein:
the one or more exposure windows comprise a first exposure window and a second exposure window;
the first exposure window is an axial opening at a distal end of the catheter, wherein at least one of the one or more lacerators is configured to lacerate the tissue when the at least one of the one or more lacerators is deployed at or from the first exposure window; and
the second exposure window is an opening in a radially outer wall of the catheter, wherein at least one of the one or more lacerators is configured to lacerate the tissue when the at least one of the one or more lacerators is deployed at or from the second exposure window.

26. The catheter of claim 21, wherein at least one of the one or more lacerators comprises a thulium fiber laser configured to pass laser energy towards the tissue at or near the one or more exposure windows to lacerate the tissue.

27. The catheter of claim 21, wherein at least one of the one or more lacerators is one or more electrodes configured to pass electrosurgical energy through the tissue at or near the one or more exposure windows to lacerate the tissue.

28. The catheter of claim 21, wherein:
the one or more aligners comprise an aligner rotatably coupled to the catheter; and
the one or more lacerators comprise a first lacerator coupled to the aligner at a distal end of the first lacerator and coupled to a control wire at a proximal end of the first lacerator, wherein the control wire is axially movable in the catheter.

29. The catheter of claim 28, wherein:
distal movement of the control wire in the catheter causes the first lacerator to be exposed through a first exposure window of the one or more exposure windows and causes rotation of the aligner away from the catheter and into a deployed configuration; and
proximal movement of the control wire in the catheter causes the first lacerator to be withdrawn into the catheter and causes rotation of the aligner toward the catheter and into a collapsed configuration.

30. The catheter of claim 29, wherein:
distal movement of the control wire in the catheter causes a first length of the first lacerator to be exposed through the first exposure window;
the aligner is configured to promote contact between the first length of the first lacerator and the tissue at or near the first exposure window for slicing the tissue with the first length of the first lacerator when the aligner is in the deployed configuration.

31. The catheter of claim 21, wherein:
the one or more exposure windows comprise a first exposure window and a second exposure window;
the first exposure window is an axial opening at a distal end of the catheter;
the second exposure window is an opening in a radially outer wall of the catheter;
the one or more aligners comprise an aligner rotatably coupled to the catheter at a position between the first exposure window and the second exposure window; and
the catheter further comprises:
a control wire axially movable in the catheter;
a first lacerator of the one or more lacerators, wherein the first lacerator is configured to be advanced through the catheter such that the first lacerator is exposable through the first exposure window to pierce the tissue; and
a second lacerator of the one or more lacerators, wherein:
a distal end of the second lacerator is coupled to the aligner;
a proximal end of the second lacerator is coupled to the control wire;
distal movement of the control wire in the catheter causes the second lacerator to be exposed through the second exposure window to slice the tissue and causes rotation of the aligner away from the catheter and into a deployed configuration in which the aligner promotes contact between the second lacerator and the tissue at or near the second exposure window; and
proximal movement of the control wire in the catheter causes the second lacerator to be withdrawn into the catheter and causes rotation of the aligner toward the catheter and into a collapsed configuration.

32. The catheter of claim 31, wherein:
the first lacerator comprises an optical fiber configured to pass laser energy towards the tissue at or near the first exposure window; and
the second lacerator comprises one or more electrodes configured to pass electrosurgical energy through the tissue at or near the second exposure window.

33. A system for lacerating a tissue, comprising:
a catheter comprising:
one or more exposure windows;
one or more lacerators, wherein the one or more lacerators are configured to lacerate the tissue and are deployed at or from the one or more exposure windows; and
one or more aligners, wherein the one or more aligners are deployable and configured to, when deployed, promote contact between the one or more lacerators and the tissue when the one or more lacerators are deployed at or from the one or more exposure windows; and
an energy source couplable to at least one of the one or more lacerators and configured to be activated to transmit energy to the tissue, the transmitted energy passing towards the tissue through the at least one of the one or more lacerators deployed at or from the one or more exposure windows.

34. The system of claim 33, wherein:
the one or more exposure windows comprise a first exposure window, wherein the first exposure window is an axial opening at a distal end of the catheter;
the one or more exposure windows comprise a second exposure window, wherein the second exposure window is an opening in a radially outer wall of the catheter;
the one or more aligners comprise an aligner rotatably coupled to the catheter; and
the catheter further comprises:
a control wire axially movable in the catheter;
a first lacerator of the one or more lacerators, wherein the first lacerator is configured to be advanced through the catheter such that the first lacerator is exposable through the first exposure window to lacerate the tissue; and
a second lacerator of the one or more lacerators, wherein:
a distal end of the second lacerator is coupled to the aligner;
a proximal end of the second lacerator is coupled to the control wire;
distal movement of the control wire in the catheter causes the second lacerator to be exposed through the second exposure window to lacerate the tissue and causes rotation of the aligner away from the catheter and into a deployed configuration in which the aligner promotes contact between the second lacerator and the tissue at or near the second exposure window; and
proximal movement of the control wire in the catheter causes the second lacerator to be withdrawn into the catheter and causes rotation of the aligner toward the catheter and into a collapsed configuration.

35. The system of claim 34, further comprising a second catheter, wherein:
the catheter is positionable through the second catheter; and
the second catheter comprises a second aligner configured to promote contact between the first lacerator and the tissue when the first lacerator is deployed at or from the first exposure window.

36. The system of claim 33, wherein:
the at least one of the one or more lacerators comprises at least one of an optical fiber or one or more electrodes, wherein:
when the at least one of the one or more lacerators comprises the optical fiber, the energy source comprises a laser source configured to transmit laser energy to the tissue, the transmitted laser energy passing through the optical fiber towards the tissue at or near the one or more exposure windows to lacerate the tissue; and
when the at least one of the one or more lacerators comprises the one or more electrodes, the energy source comprises an electrosurgical energy source configured to transmit electrosurgical energy to the tissue, the transmitted electrosurgical energy passing from or between the one or more electrodes through the tissue at or near the one or more exposure windows to lacerate the tissue.

37. A method, comprising:
advancing a catheter toward a tissue;
piercing the tissue at or near a first exposure window of the catheter to generate an opening in the tissue;
advancing at least a portion of the catheter through the opening in the tissue;
deploying one or more aligners to promote contact between a lacerator and the tissue when the lacerator is deployed at or from a second exposure window of the catheter; and
slicing the tissue at or near the second exposure window of the catheter.

38. The method of claim 37, further comprising advancing a control wire distally through the catheter, wherein advancing the control wire distally:
exposes the lacerator through the second exposure window to slice the tissue; and
rotates a first aligner of the one or more aligners away from the catheter from a collapsed configuration to a deployed configuration in which the first aligner promotes contact between the lacerator and the tissue when the lacerator is exposed through the second exposure window.

39. The method of claim 38, wherein:
advancing the control wire distally exposes a first length of the lacerator through the second exposure window; and
rotation of the first aligner of the one or more aligners away from the catheter promotes contact between the first length of the lacerator and the tissue.

40. The method of claim 38, further comprising deploying a second aligner of the one or more aligners to promote positioning of the catheter with respect to the tissue for piercing of the tissue at or near the first exposure window.
